(19) United States
(12) Patent Application Publication
(10) Pub. No.: US 2020/0060525 A1
(43) Pub. Date: Feb. 27, 2020

(54) Methods and Medical Devices for Diverticulosis Treatment

(71) Applicants: Jillian Noblet, Bloomington, IN (US); Joshua Krieger, Topsfield, MA (US); Sean Chambers, Bloomington, IN (US); Gary L. Neff, Bloomington, IN (US); Tyler J Marquardt, Genoa, OH (US); Bhavesh Patel, San Diego, CA (US); Ghassan Kassab, La Jolla, CA (US)

(72) Inventors: Jillian Noblet, Bloomington, IN (US); Joshua Krieger, Topsfield, MA (US); Sean Chambers, Bloomington, IN (US); Gary L. Neff, Bloomington, IN (US); Tyler J Marquardt, Genoa, OH (US); Bhavesh Patel, San Diego, CA (US); Ghassan Kassab, La Jolla, CA (US)

(21) Appl. No.: 16/551,016

(22) Filed: Aug. 26, 2019

Related U.S. Application Data
(60) Provisional application No. 62/723,138, filed on Aug. 27, 2018.

(51) Int. Cl. A61B 1/015; A61B 1/31; A61M 3/02; A61B 17/22

(52) U.S. Cl. CPC A61B 1/015; A61B 1/31; A61B 5/0084; A61B 17/22; A61M 3/0295

(57) ABSTRACT

Methods of treating diverticulosis and medical devices suitable for use in such methods are described herein. In an embodiment, a method of treating diverticulosis in an animal comprises clearing the lumen of the colon of the animal of solid matter, isolating a treatment portion of the colon, applying suction at a point within the lumen of the colon to depressurize the treatment portion, applying a material to the external surface across at least one diverticulum of the diverticula on the colon, and removing the suction. In some embodiments, the material comprises an adhesive. A step of applying a second material across at least one diverticulum of the diverticula on the colon can also be included.

### CROSS-REFERENCE TO RELATED APPLICATIONS

This application claims the benefit of U.S. Provisional Patent Application No. 62/723,138, filed on Aug. 27, 2018. This related application is incorporated by reference into this disclosure in its entirety.

### FIELD

The disclosure relates generally to the field of methods of treatment, medical devices, and kits. Particular examples in the disclosure relate to methods of treating diverticulosis, medical devices useful in the treatment of diverticulosis, and kits useful in the treatment of diverticulosis.

### BACKGROUND

Diverticulosis of the colon is the most frequent anatomical colon alteration, characterized by the presence of pockets called ‘diverticula’ Diverticula result from a herniation of the colonic mucosa and submucosa through defects in the muscle layer, often at the weakest point in the colonic wall: the sites of penetration by blood vessels. Approximately 50% of individuals aged 60+ will have diverticulosis and incidence increases to 70% of individuals aged 80+. Diverticulosis is becoming increasingly common and occurring in younger patients, often presenting with more substantial complications. The presence of complications such as inflammation, diverticular bleeding, infection, perforation and sepsis characterize the so-called ‘diverticular disease’ or ‘diverticulitis.’

Uncomplicated diverticulosis (asymptomatic) identified on colonoscopy is currently treated with a high-fiber, low fat diet and an increase in physical activity. However, clinical studies have failed to demonstrate efficacy of this treatment approach. Complicated diverticulosis is currently treated with intravenous antibiotics and surgical resection of the affected region of the colon. Several patient populations would benefit from minimally invasive approaches to treatment, include patients that are at high risk for surgery and those which are indicated for surgery in the complicated diverticulosis group.

A need exists, therefore, for new and useful methods of treatment, medical devices, and kits. Furthermore, a specific need exists for new and useful methods of treating diverticulosis, medical devices useful in the treatment of diverticulosis, and kits useful in the treatment of diverticulosis.

### SUMMARY OF SELECTED EXAMPLE EMBODIMENTS

Various methods of treatment, medical devices, and kits are described herein.

An example method of treating diverticulosis comprises clearing the lumen of a colon of solid matter; isolating a treatment portion of the colon, the treatment portion having an axial length; applying suction at a point within the lumen to depressurize the treatment portion; applying a material across at least one diverticulum on the external surface of the colon, the at least one diverticulum located on a surface of the colon within the axial length of the treatment portion; and removing the suction.

Another example method of treating diverticulosis comprises clearing the lumen of a colon of solid matter; isolating a treatment portion of the colon, the treatment portion having an axial length; applying suction at a point within the lumen to depressurize the treatment portion; applying an adhesive across two or more diverticuli on the external surface of the colon, the two or more diverticuli located on a surface of the colon within the axial length of the treatment portion; applying a material across the two or more diverticuli; curing the adhesive; and removing the suction.

Another example method of treating diverticulosis comprises clearing the lumen of a colon of solid matter by administering an enema; isolating a treatment portion of the colon by inserting a catheter having a balloon into the colon and inflating the balloon within the lumen to isolate the treatment portion from another portion of the colon, the treatment portion having an axial length; applying suction at a point within the lumen to depressurize the treatment portion; applying an adhesive across two or more diverticuli on the external surface of the colon, the two or more diverticuli located on the surface of the colon within the axial length of the treatment portion; applying a material across the two or more diverticuli; curing the adhesive; and removing the suction.

Additional understanding of the claimed invention can be obtained through review of the detailed description of selected example medical devices, methods of treatment, and kits, below, with reference to the appended drawings.

### BRIEF DESCRIPTION OF THE DRAWINGS

FIG. 1 is a schematic illustration of an example method of treating diverticulosis.

FIG. 2A is a schematic illustration of an isolated section of a colon upon which the method of treatment illustrated in FIG. 1 is being performed.

FIG. 2B is a schematic illustration of an isolated section of a colon upon which the method of treatment illustrated in FIG. 1 is being performed.

FIG. 2C is a schematic illustration of an isolated section of a colon upon which the method of treatment illustrated in FIG. 1 is being performed.

FIG. 2D is a schematic illustration of an isolated section of a colon upon which the method of treatment illustrated in FIG. 1 is being performed.

FIG. 3 is a schematic illustration of another example method of treating diverticulosis.

FIG. 4 is a schematic illustration of another example method of treating diverticulosis.

FIG. 5 is a perspective view of an example medical device.

FIG. 6 is a sectional view, partially broken away, of the medical device illustrated in FIG. 5. The inner member is illustrated in a first position.

FIG. 7 is a sectional view, partially broken away, of the medical device illustrated in FIG. 6. The inner member is illustrated in a second position.

FIG. 8 is a perspective view of the inner member of the medical device illustrated in FIG. 5.

FIG. 9 is a perspective view, partially broken away, of a section of a colon within which the medical device illustrated in FIG. 5 is partially disposed.

FIG. 10 is a side view of another example medical device.

FIG. 11A illustrates an example medical device.

FIG. 11B illustrates an example medical device.

FIG. 11C illustrates an example medical device.

FIG. 11D illustrates an example medical device.

FIG. 12A illustrates an example medical device.

FIG. 12B illustrates an example medical device.

FIG. 12C illustrates an example medical device.

FIG. 13 illustrates an example medical device.

FIG. 14 illustrates an example medical device.

FIG. 15 illustrates an example medical device.

### DETAILED DESCRIPTION OF SELECTED EXAMPLES

The following detailed description and the appended drawings describe and illustrate various example medical devices, methods of treatment, and kits. The description and illustration of these examples are provided to enable one skilled in the art to make and use a medical device according to an embodiment of the invention, to practice a method of treatment according to an embodiment of the invention, and to make and use a kit according to an embodiment of the invention.

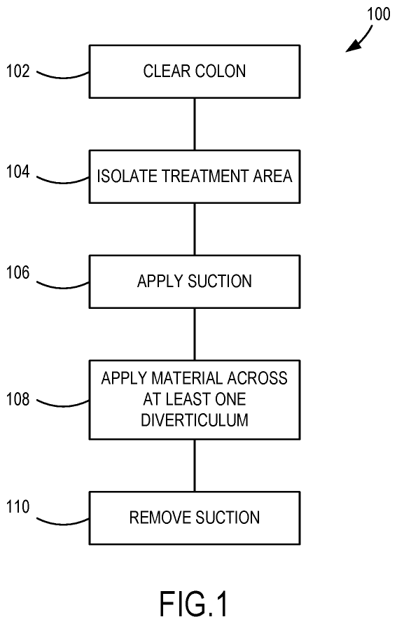

FIG. 1 is a schematic illustration of an example method 100 of treating diverticulosis. An initial step 102 comprises clearing the lumen of the colon of solid matter. Another step 104 comprises isolating a treatment portion of the colon. Another step 106 comprises applying suction at a point within the central lumen of the colon to depressurize the treatment portion isolated in step 104. Another step 108 comprises applying a material to the external surface of the colon across at least one diverticulum that is located on a surface of the colon within the axial length of the treatment portion isolated in step 104. Another step 110 comprises removing the suction applied in step 106.

The step 102 of clearing the lumen of the colon of solid matter can be accomplished using any suitable method or technique for clearing contents from an animal colon. For example, an enema or other clearance aid can be used to accomplish this step 102. For this step 102, it is considered sufficient to substantially clear the colon of solid matter; complete and absolute clearance is not required. This step 102 should be performed to achieve a clearance suitable for performance of the remainder of the method. For example, a clearance that does not impede the placement of a device used in step 106 to apply suction at a point with the central lumen of the colon and that does not impede the depressurization of the treatment are and any isolated diverticuli is considered sufficient.

The step 104 of isolating a treatment portion of the colon can be accomplished using any suitable method or technique and, if desired, any suitable device. For example, one or more balloons can be expanded within the central lumen of the colon to isolate a treatment portion. For this step, the term “isolate” does not require the establishment of a physical barrier between the treatment portion and other portion or portions of the colon. Rather, the term merely requires the establishment or identification of a demarcation between an area of the colon for treatment and another area of the colon. The establishment of a physical barrier between a desired treatment portion and other portions of the colon, by expanding balloons within the colon, for example, is one suitable approach for accomplishing this step 104, but this approach is not required. If the establishment of one or more physical barriers is desired, use of a medical device is considered advantageous, and a variety of medical devices can be used. Examples of suitable medical devices for performance of this step include, but are not limited to, a single balloon catheter, a dual balloon catheter, an occluder, and a covered stent. Each of these devices can be delivered intraluminally within the colon and deployed appropriately at a desired location within the lumn of the colon to achieve the desired isolation. External devices, such as clips, can also be used to achieve the desired isolation.

The step 106 of applying suction at a point within the central lumen of the colon to depressurize the treatment portion isolated in step 104 can be accomplished using any suitable method or technique and, if desired, any suitable device. For example, the proximal end of a catheter or other tubular member can be operably connected to a suction source at one end, external to the body of the patient on which the method is being performed. The distal end of the catheter can be placed within the treatment portion isolated in step 104. Once arranged in this manner, the suction source can be activated such that suction is applied at the distal end of the catheter, within the central lumen of the colon to depressurize the treatment portion isolated in step 104. Importantly, for this step 106, it is important that suction be applied within the central lumen of the colon and not within the interior portion of any individual diverticuli. The method 100 is optimized for treating any suitable number of diverticuli, including more than one diverticulum. Accordingly, application of suction at a point within the central lumen of the colon is considered an important aspect of this step of the method 100.

The step 106 of applying suction at a point within the central lumen of the colon to depressurize the treatment portion isolated in step 104 is performed to relieve residual stress in one or more diverticuli associated with the treatment portion. Accordingly, this step 106 of applying suction should be performed until one or more diverticuli associated with the treatment portion loses residual stress, which may result in collapse or other decrease in overall size, and/or volume. A visual observation of one or more diverticuli can be used to determine that the desired relief of residual stress in one or more diverticuli has been achieved, but is not considered necessary.

The step 106 of applying suction can be performed for any suitable length of time, and can be maintained while other steps are be performed. For example, in one example method, the step 106 of applying suction is performed until at least one diverticulum associated with the treatment portion isolated in step 102 has been relieved of residual stress. Once that is achieved, in this example, the step 110 of removing suction is performed. At this point, the treatment portion of the colon, and the at least one diverticulum, is under negative pressure compared to the normal pressure for these elements. At this point, the step 108 of applying a material across at least one diverticulum can be performed. In another example method, the step 106 of applying suction is performed until at least one diverticulum associated with the treatment portion isolated in step 102 has been relieved of residual stress. At this point, the step 106 of applying suction is continued while the step 108 of applying a material across at least one diverticulum is performed. In this example, the step 110 of removing suction is not performed until after the step 108 of applying a material across at least one diverticulum is completed.

The step 108 of applying a material to the external surface of the colon across at least one diverticulum that is located on a surface of the colon within the axial length of the treatment portion isolated in step 104 can be accomplished using any suitable method or technique and any suitable material. For example, a patch of material can be applied to the serosal or external surface of the colon across at least one diverticulum that is located on a surface of the colon within the axial length of the treatment portion isolated in step 104. For this step 108, the material can be applied to the external surface of the colon across at least one diverticulum solely through an external approach, such as by surgical, laparoscopic, or other access. Alternatively, the material can be applied to the external surface of the colon across at least one diverticulum through a combination of internal and external approaches. For example, an adhesive can be delivered to the external location from within the colon and a patch of material can then be applied directly onto the adhesive through an external approach.

For this step 108, any suitable material can be used and a skilled artisan will be able to select a specific material for a particular method based on various considerations, including the overall size and configuration of the treatment portion and the number of diveritculi being treated. A material should be selected based on biocompatibility and handling considerations, as well as the ability of the material to decrease the load placed on the colon wall. Examples of suitable types of materials include, but are not limited to, hydrogels, remodellable materials, such as extracellular matrix materials, and other materials. Examples of specific materials considered suitable include, but are not limited to, small intestinal submucosa (SIS), pulmonary ligament, visceral pleura, pericardium, and peritoneum. It is noted that more than one material can be applied in connection with performance of this step 108. For example, for some materials, it may be desirable to apply an adhesive prior to applying a patch of material to the external surface of the colon. Also, a method can include additional steps based on the nature of the material or materials selected for this step. For example, if an adhesive is used, a suitable curing step can be performed. Example methods that include application of an adhesive are described in more detail below.

It is considered advantageous to perform this step 108 of applying a material to the external surface of the colon across at least one diverticulum that is located on a surface of the colon within the axial length of the treatment portion isolated in step 104 after the step 106 of applying suction has been initiated or completed. It is noted, though, that this step 108 can be performed prior to the initial of step. Thus, step 108 can be performed before, during, or after initiation of step 106. Furthermore, step 108 can be performed concurrently with step 106.

The step 110 of removing the suction applied in step 106 can be accomplished using any suitable method or technique and the specific method or technique selected in a particular method will depend on the suction source and device selected for performance of step 106 of applying suction. For example, this step 110 of removing the suction applied in step 106 can be accomplished by deactivating the suction source activated during performance of that step 106. The step 110 of removing the suction applied in step 106 is performed to restore a normal pressure within the colon.

It is considered advantageous to perform this step 110 of removing the suction applied in step 106 after step 108 has been completed. It is noted, though, that this step 110 can be performed prior to the initial of step 108 or during performance of step 108. Thus, step 110 can be performed before, during, or after initiation of step 108. Furthermore, step 110 can be performed concurrently with step 108.

The method 100 can include various optional steps. For example, a step of applying positive pressure with the abdominal cavity, external to the colon, can be performed. Including this step can supplement the relief of residual stress on diverticuli achieved by the application of suction within the colon. If included, the step is advantageously performed before the step 108 of applying a material to the external surface of the colon across at least one diverticulum that is located on a surface of the colon within the axial length of the treatment portion isolated in step 104.

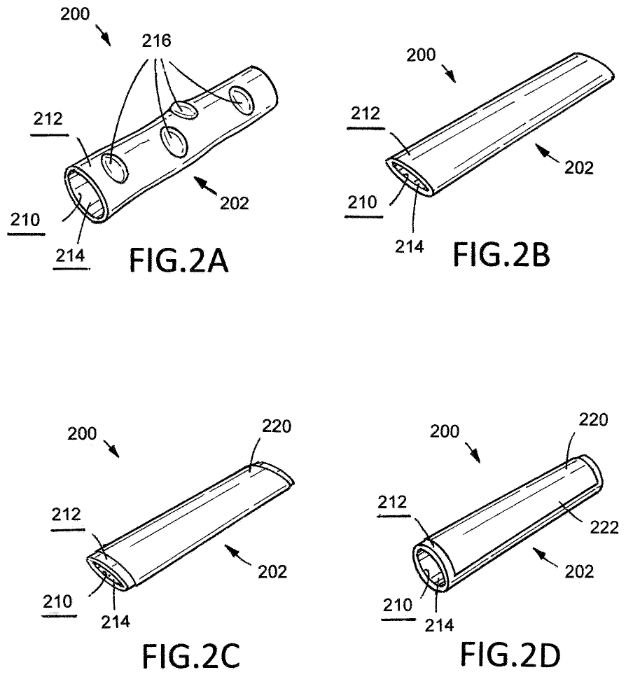

Each of FIGS. 2A, 2B, 2C, and 2D illustrates an isolated section of a colon 200 upon which the example method 100 illustrated in FIG. 1 and described above is being performed. The colon 200 has an internal surface 210, or mucosal surface, and an external surface 212, or serosal surface, and an internal lumen 214. Several diverticuli 216 are visible on the external surface 212 of the colon 200. Each of FIGS. 2A, 2B, 2C, and 2D only shows a portion of the colon 200; for purposes of illustration, the illustrated portion is considered the isolated portion 202 of the colon 200.

FIG. 2A illustrates the colon 200 after completion of steps 102 and 104 of method 100. The colon 200 been cleared of solid matter such that the internal lumen 214 is substantially free of fecal material.

FIG. 2B illustrates the colon 200 while step 106 of method 100 is being performed. As such, suction has been applied at a point within the internal lumen 214 of the colon 200 to depressurize the treatment portion 202. The colon 200 has flattened, with its cross-sectional shape changing from substantially circular to substantially ovoid. Also, the diverticuli 216 associated with the treatment portion 202 have lost residual stress as a result of the application of suction within the internal lumen 214 of the colon 200. Indeed, in FIG. 2B, each of the diverticuli 216 has collapsed such that the surface of the diverticuli 216 is substantially continuous with the adjacent portion of the external surface 212 of the colon 200.

FIG. 2C illustrates the colon 200 after step 108 of method 100 has been performed. Thus, a patch of material 220 has been applied to the external surface 212 of the colon 200. The patch of material 220 has been applied across the diverticuli 216 associated with the treatment portion 202.

FIG. 2D, illustrates the colon 200 after step 110 of method 100 has been performed. Thus, suction has been removed and normal pressure has been restored with the internal lumen 214 of the colon 200. Accordingly, the colon 200 has returned to its normal configuration, having a substantially circular cross-sectional shape. Notably, the bulges of the diverticuli 216 visible in FIG. 2A have not returned and the patch of material 220 presents a new, substantially continuous external surface 222 for the treatment portion 202 of the colon 200.

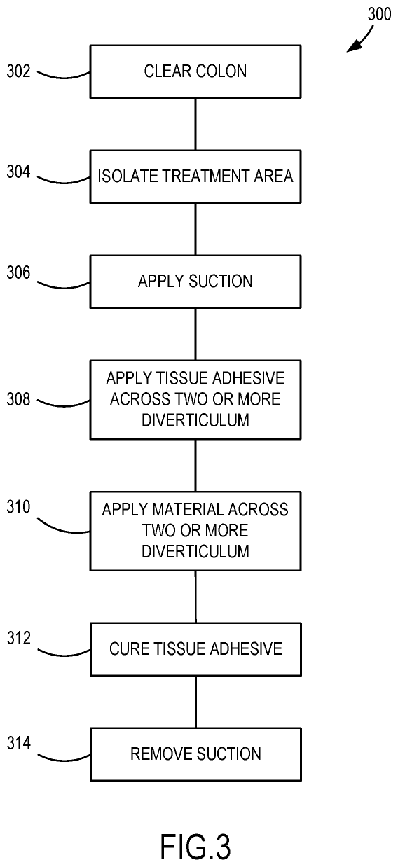

FIG. 3 is a schematic illustration of another example method 300 of treating diverticulosis. An initial step 302 comprises clearing the lumen of the colon of solid matter. Another step 304 comprises isolating a treatment portion of the colon. Another step 306 comprises applying suction at a point within the central lumen of the colon to depressurize the treatment portion isolated in step 304. Another step 308 comprises applying tissue adhesive across two or more diverticuli that are located on a surface of the colon within the axial length of the treatment portion isolated in step 104. Another step 310 comprises applying a material to the external surface of the colon across the two or more diverticuli. Another step 312 comprises curing the tissue adhesive. Another step 314 comprises removing the suction applied in step 106.

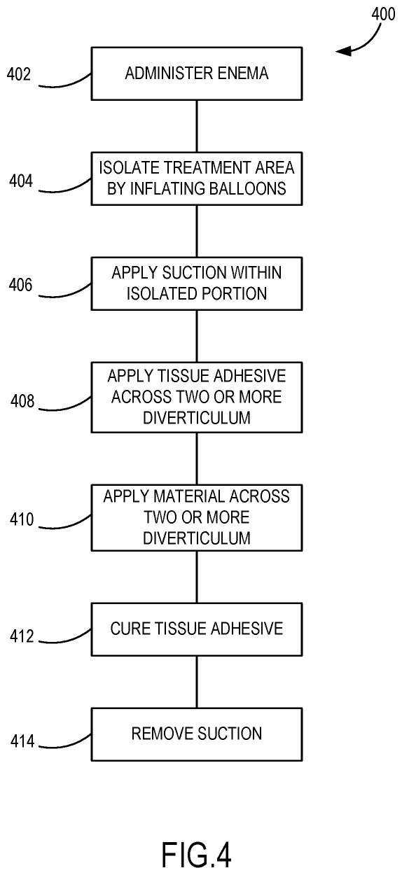

FIG. 4 is a schematic illustration of another example method 400 of treating diverticulosis. An initial step 402 comprises administering an enema to a patient to clear the patient's colon of solid matter. Another step 404 comprises isolating a treatment portion of the colon by inflating balloons within the colon. Another step 406 comprises applying suction at a point within the central lumen of the colon to depressurize the treatment portion isolated in step 404. Another step 408 comprises applying tissue adhesive across two or more diverticuli that are located on a surface of the colon within the axial length of the treatment portion isolated in step 404. Another step 410 comprises applying a material to the external surface of the colon across the two or more diverticuli. Another step 412 comprises curing the tissue adhesive. Another step 414 comprises removing the suction applied in step 406.

FIGS. 5 through 9 illustrate an example medical device 500 useful in treating diverticulosis to a patient. Medical device 500 comprises an elongate member 510 having an elongate member first end 522, an elongate member second end 524, a lengthwise axis 511 extending between the elongate member first end 522 and the elongate member second end 524, a length 523 extending between the elongate member first end 522 to the elongate member second end 524, a diameter 525, and an elongate member main body 526 extending between the elongate member first end 522 and the elongate member second end 524. The elongate member main body 526 defines a first lumen 512, a second lumen 514, a suction lumen 516, an elongate member circumferential wall 528, a plurality of passageways 530, an inner surface 532 extending between the elongate member first end 522 and the elongate member second end 524, and an outer surface 534 extending between the elongate member first end 522 and the elongate member second end 524. Additionally, the medical device 500 comprises an inner member 520 disposed in the elongate member 510, as described in more detail below.

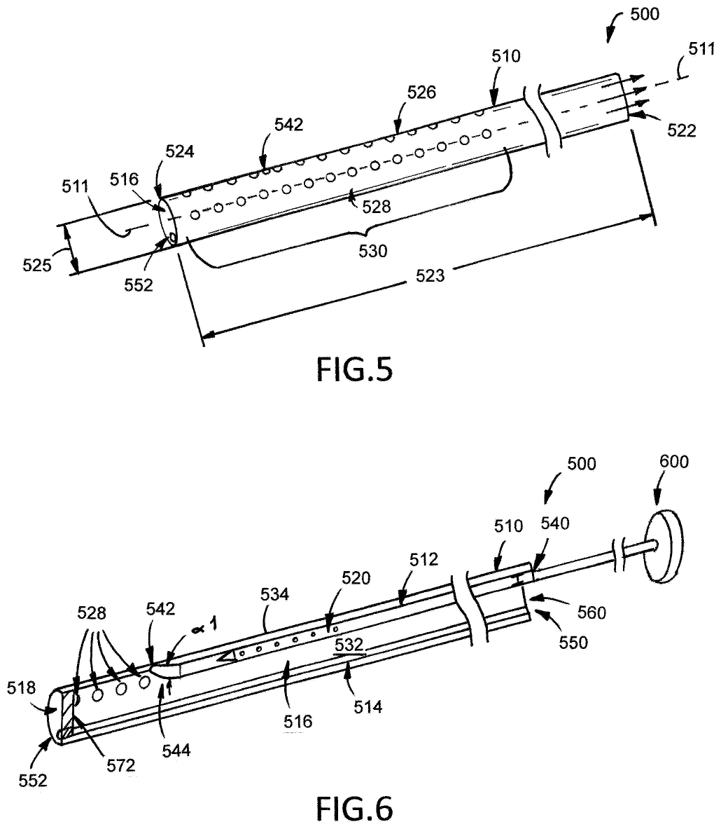

First lumen 512 extends from a first lumen first opening 540 to a first lumen second opening 542 and is disposed on the inner surface 532 of the elongate member 510 extending along the lengthwise axis 511 of the elongate member 510 between the elongate member first end 522 toward the elongate member second end 524. As best illustrated in FIG. 6, the first lumen first opening 540 is disposed on the elongate member first end 522, and the first lumen second opening 542 is disposed between the elongate member first end 522 and the elongate member second end 524 toward the elongate member second end 524. Second lumen 514 extends from a second lumen first opening 550 to a second lumen second opening 552 and is disposed on the inner surface 532 of the elongate member 510 extending along the lengthwise axis 511 of the elongate member 510 between the elongate member first end 522 to the elongate member second end 524. As best illustrated in FIG. 6, the second lumen first opening 550 is disposed on the elongate member first end 522, and the second lumen second opening 552 is defined by the cap 518, as described in detailed below, such that the second lumen second opening 552 is continuous with the second lumen 514. The first and second lumens 512, 514 are parallel to each other because each lumen 512, 514 lies in parallel with the lengthwise axis 511 of the elongate member 510, but the first and second lumens 512, 514 are substantially opposite of each other. Furthermore, the first and second lumens 512, 514 of the elongate member 510 are isolated from each other such that the first and second lumen 512, 514 are not in fluid communication.

The inclusion of the first lumen 512 is considered advantageous at least because the first lumen 512 allows for the introduction of a device through the elongate member 510 that is isolated from the second lumen 514 and the suction lumen 516. Any suitable device can be passed through a first lumen, and skilled artisans will be able to select a suitable device according to a particular embodiment based on various considerations, including the desired treatment intended to be performed. Example devices considered suitable to pass through a first lumen defined by an elongate member include, but are not limited to, catheters, cannulas, suction devices, cutting tools, illuminating members, optical fibers, and any other devices considered suitable for a particular application. For example, an inner member, such as a cannula, can be passed through a first lumen of an elongate member that comprises an atraumatic tip and a plurality of passageways, as described in more detail below.

The inclusion of the second lumen 514 is considered advantageous at least because the second lumen 514 allows for the introduction of a device through the elongate member 510 that is isolated from the first lumen 512 and the suction lumen 516. Any suitable device can be passed through a second lumen, and skilled artisans will be able to select a suitable device according to a particular embodiment based on various consideration, including the desired treatment intended to be performed. Example devices considered suitable to pass through a second lumen defined by an elongate member include, but are not limited to, catheters, cannulas, suction devices, cutting tools, illuminating members, optical fibers, and any other devices considered suitable for a particular application. For example, a device, such as a balloon catheter including one or more balloons, can be passed through a second lumen of an elongate member that comprises one or more balloons to isolate a treatment portion of a colon.

Each of the first lumen first opening 540, first lumen second opening 542, second lumen first opening 550, and second lumen second opening 552 can have any suitable size, shape, and configurations, and a skilled artisan will be able to select suitable size, shape, and configuration parameters for an opening in an elongate member of a medical device according to a particular embodiment based on various considerations, including the size of a device passing toward and through the openings, the shape of a device passing toward and through the openings, and other considerations. Additionally, each of the first lumen first opening 540, first lumen second opening 542, second lumen first opening 550, and second lumen second opening 552 can have any suitable structural configuration. Examples of suitable structural configuration include, but are not limited to, circular, substantially circular, ovoid, elliptical, and any other suitable structural configuration. In the illustrated embodiment, each of the first lumen first opening 540, first lumen second opening 542, second lumen first opening 550, and second lumen second opening 552 illustrates a circular shape.

While the first lumen first opening 540, first lumen second opening 542, second lumen first opening 550, and second lumen second opening 552 have been described and illustrated as positioned at particular locations along the elongate member 510 of the medical device 500, an opening of a lumen can be positioned at any suitable location on, or along the length of, an elongate member of a medical device. Skilled artisans will be able to select a suitable location to position a lumen opening according to a particular embodiment based on various considerations, including the desired bodily passage within which a medical device is intended to be used. Example positions considered suitable to locate an opening of a lumen on an elongate member include, but are not limited to, on an elongate member first end, along the length of an elongate member between an elongate member first end and an elongate member second end, and an elongate member second end. For example, alternative to positioning a first lumen second opening along the length of an elongate member between an elongate member first end and an elongate member second end, a first lumen second opening can be positioned on an elongate member second end. Additionally, alternative to the positioning a second lumen second opening on an elongate member second end, a second lumen second opening can be positioned along a length of an elongate member between an elongate member first end and an elongate member second end.

While the elongate member 510 has been described and illustrated with a length 523 and a diameter 525, an elongate member can comprise any suitable length and diameter for a medical device. Skilled artisans will be able to select a suitable length and diameter according to a particular embodiment based on various considerations, including the length and width of the desired bodily passage within which a medical device is intended to be used. An example length for an elongate member considered suitable comprises a length between about 1.0 meter to about 1.5 meters, and an example diameter for an elongate member considered suitable comprises a diameter between about 0.8 centimeter to about 2.0 centimeters.

Furthermore, as best illustrated in FIGS. 6 and 7, the first lumen 512 defines a bend 544 that extends from the first lumen first opening 540 toward the first lumen second openings 542. In the illustrated embodiment, the bend 544 defines a first angle α₁ relative to the lengthwise axis 511 of the elongate member 510. The inclusion of the bend 544 is considered advantageous at least because the bend 544 guides a device, such as an inner member, by axially advancing it in a direction toward the elongate member second end 522 within the first lumen 512 of the elongate member 510 toward the first lumen second opening 542. As such, the bend 544 is generally aligned with the first lumen second opening 542. Indeed, the bend 544 is continuous with the first lumen second opening 542. A bend can have any suitable size, shape, and configuration, and a skilled artisan will be able to select suitable size, shape and configuration parameters for a bend in a medical device according to a particular embodiment based on various considerations, including the material from which the elongate member of the subject medical device is formed, the nature and flexibility of any device intended to be advanced within the first lumen of the elongate member and through the first lumen second opening of the subject medical device, and other considerations. In the illustrated embodiment, the bend 544 is integrally formed with the first lumen 512. Also in the illustrated embodiment, the bend 544 is of a curvilinear shape. The inclusion of the curvilinear shape, or a bend defining another suitable curve, is considered advantageous at least for use with a flexible device, such as a cannula, because the bend can effectively guide such secondary member toward and through the first lumen second opening 542. For a stiffer device, a substantially flat, planar, or non-curved bend is considered advantageous at least because such bends can effectively guide such stiffer devices toward and through the first lumen second opening 542.

Suction lumen 516 is defined by the inner surface 532 of the elongate member 510 and extends from the suction lumen first opening 560 and terminates at the elongate member second end 522 along a lengthwise axis 511 of the elongate member 510. In the illustrated embodiment, the suction lumen 516 is in fluid communication with each passageway from the plurality of passageways 530 of the elongate member 510, as described in detail below. The fluid communication between the suction lumen 516 and each passageway of the plurality of passageways 530 is considered advantageous at least because it allows the suction lumen 516 to equally depressurize a treatment portion of a colon, such as treatment portion 202 of colon 200 illustrated in FIGS. 2A, 2B, 2C, and 2D, by distributing equal suction to each passageway in the plurality of passageways 530 along the lengthwise axis 511 of the elongate member 510. Additionally, as best illustrated in FIG. 6, the first and second lumen 512, 514 are disposed in the suction lumen 516, but each of the first and second lumens 512, 514 is isolated from the suction lumen 516. The isolation between each of the first lumen 512, the second lumen 514, and the suction lumen 516 is considered advantageous at least because it allows a user, such as surgeon, to introduce suction to depressurize a treatment portion 202 of a colon 200 while introducing a device into the first lumen 512 and/or a device into the second lumen 514 simultaneously.

In the illustrated embodiment, the elongate member circumferential wall 528 defines a plurality of passageways 530 that individually provide fluid communication between the suction lumen 516 and an environment external to the medical device. Each passageway of the plurality of passageways 530 extends between the inner surface 532 of the elongate member main body 526 to the outer surface 534 of the elongate member main body 526. As best illustrated in FIG. 5, the plurality of passageways 528 is disposed on a side along a portion of the elongate member 510 extending between the elongate member second end 524 toward the elongate member first end 522. While the illustrated embodiment includes a plurality of passageways 528, any suitable number of passageways can be included. Moreover, the passageways in a medical device according to a particular embodiment can have any suitable size, shape, configuration, arrangement, and location. The number, size, shape, configuration, arrangement, and location of passageways included in a medical device according to a particular embodiment can be selected by a skilled artisan on various considerations, including the nature of the colon within which the medical device is intended to be used, the size of the diverticulum being treated, and other considerations. In the illustrated embodiment, the plurality of passageways comprises equally positioned series of passageways that are disposed on a side along a portion of the elongate member toward the elongate member second end to facilitate distribution of suction to collapse a treatment portion of a colon and two or more diverticula simultaneously by use of an external suction source. It is noted, though, the plurality of passageways 528 may also comprise randomly positioned series of passageways disposed circumferentially about a portion of the elongate member toward the elongate member second end to facilitate distribution of suction to collapse a treatment portion of a colon and two or more diverticula simultaneously by use of an external suction source.

While the plurality of passageways 530 of the elongate member 510 has been described and illustrated as being located on a portion of the elongate member 510 toward the elongate member second end 522, a plurality of passageways can be positioned at any suitable location on, or along the length of, an elongate member of a medical device. Skilled artisans will be able to select a suitable location to position a plurality of passageways according to a particular embodiment based on various considerations, including the length of the treatment portion of the colon within which a medical device is intended to be used. Example positions considered suitable to locate a plurality of passageways on an elongate member include, but are not limited to, on the elongate member first end, along the length of an elongate member between the elongate member first end and the elongate member second end, and the elongate member second end. For example, alternative to positioning a plurality of passageways on the elongate member second end, the plurality of passageways can be positioned along the length of an elongate member between an elongate member first end and an elongate member second end. Additionally, the inventors have determined that examples of suitable lengths for a plurality of passageways of an elongate member includes lengths between about 10 centimeters to about 40 centimeters.

While each passageway of the plurality of passageways 528 of the elongate member 510 has been described and illustrated in the illustrated embodiment, each passageway of a plurality of passageways can have any suitable structural configuration according to an embodiment. Skilled artisans will be able to select a suitable structural configuration for each passageway of a plurality of passageways according to a particular embodiment based on various considerations, including the size of the treatment portion of the affected colon. Examples of suitable structural configurations for each passageway of a plurality of passageways include, but are not limited to, circular, substantially circular, ovoid, elliptical, and any other suitable structural configuration. Additionally, the inventors have determined that examples of a suitable diameter for each passageway of a plurality of passageways includes diameters between about 0.10 centimeter to about 1.0 centimeter.

Cap 518 is disposed on the elongate member second end 524 and defines the second lumen second opening 552. As illustrated in FIGS. 5 through 7, the cap 518 is attached to the elongate member 518 and defines the elongate member second end 524. The cap 518 also defines a terminating surface 572 such that when the cap 518 is attached to the elongate member 510, the terminating surface 572 seals and terminates the suction lumen 516. In the illustrated example, the cap 518 is integrally formed with the elongate member 510 on the elongate member second end 524. Examples of suitable attachment between the cap 518 and the elongate member 510 include fixed attachment formed with an adhesive bond between the elongate member 510 and the cap 518 and a mechanical connection formed between the elongate member 510 and the cap 518.

The elongate member 510 can be formed of any suitable flexible, or substantially flexible, material, and skilled artisans will be able to select a suitable material for an elongate member according to a particular embodiment based on various considerations, including the bodily passage within which the elongate member is intended to be used. Example materials considered suitable include, but are not limited to, plastics, metals, and other materials used in conventional medical devices.

FIG. 8 illustrates an example inner member 520 disposed within the elongate member 510. Inner member 520 comprises an inner member first end 580, an inner member second end 582, a length 581 extending between the inner member first end 580 and the inner member second end 582, a diameter 583, and an inner member main body 584 extending between the inner member first end 580 and the inner member second end 582. The inner member main body 584 defines an inner member interior lumen 586 extending between the inner member first end 580 to the inner member second end 582, an inner member circumferential wall 588, a plurality of passageways 590, an atraumatic tip 592, and an outer surface 594 extending between the inner member first end 580 to the inner member second end 582.

In the example embodiment, the inner member circumferential wall 588 defines a plurality of passageways 590 that individually provide fluid communication between the inner member interior lumen 586 and an environment external to the inner member. Each passageway of the plurality of passageways 590 extends between the outer surface 594 of the inner member main body 584 and the inner member interior lumen 586 of the inner member main body 584. As best illustrated in FIGS. 7 through 9, the plurality of passageways 590 is disposed along a portion of the inner member 520 extending between the inner member second end 582 toward the inner member first end 580. While the illustrated embodiment includes a plurality of passageways 590, any suitable number of passageways can be included. Moreover, the passageways in an inner member according to a particular embodiment can have any suitable size, shape, configuration, arrangement, and location. The number, size, shape, configuration, arrangement, and location of passageways included in an inner member according to a particular embodiment can be selected by a skilled artisan on various considerations, including the nature of the colon within which the inner member is intended to be used, the size of the diverticulum region being treated, and other considerations. In the illustrated embodiment, the plurality of passageways 590 comprises equally positioned series of passageways that are disposed on a side of the inner member 520 toward to the inner member second end 582. The plurality of passageways 582 are used to facilitate distribution of adhesive by spraying the adhesive onto two or more diverticula at a treatment portion of the colon once suction is applied to the medical device. It is noted, though, the plurality of passageways 582 may also comprise a randomly positioned series of passageways disposed circumferentially about a portion of the inner member 520 toward the inner member second end 582. Furthermore, examples of suitable structural configuration for each passageway of the plurality of passageways include, but are not limited to, circular, substantially circular, ovoid, elliptical, and any other suitable structural configuration.

While the plurality of passageways 590 of the inner member 520 has been described and illustrated as being located on a portion of the inner member 520 toward the inner member second end 582, a plurality of passageways of an inner member can be positioned at any suitable location on, or along the length of, an inner member of the medical device. Skilled artisans will be able to select a suitable location to position a plurality of passageways according to a particular embodiment based on various considerations, including the length of the treatment portion of the affected colon and the size of the diverticulum in which a medical device is intended to be used. Example positions considered suitable to locate a plurality of passageways on an inner member include, but are not limited to, on the inner member first end, along the length of an inner member between the inner member first end and the inner member second end, and the inner member second end. For example, alternative to positioning a plurality of passageways on the inner member second end, the plurality of passageways can be positioned along the length of an inner member between an inner member first end and an inner member second end. Additionally, the inventors have determined that examples of suitable lengths for a plurality of passageways of an inner member includes lengths between about 10 centimeters to about 30 centimeters.

While each passageway of the plurality of passageways 528 has been described and illustrated in the illustrated embodiment, the plurality of passageways of the inner member can have any suitable structural configuration according to an embodiment. Skilled artisans will be able to select a suitable structural configuration for each passageway of a plurality of passageways of an inner member according to a particular embodiment based on various considerations, including the size of the treatment portion of the colon. Examples of suitable structural configurations for each passageway of the plurality of passageways of an inner member include, but are not limited to, circular, substantially circular, ovoid, elliptical, and any other suitable structural configuration. Additionally, the inventors have determined that examples of suitable diameter for each passageway of a plurality of passageways of an inner member includes diameters between about 0.10 millimeter to about 0.5 millimeters.

Inner member interior lumen 586 extends from an inner member interior lumen first opening 594, which is positioned on the inner member first end 580, and terminates at the inner member second end 582. The inner member interior lumen 586 is in fluid communication with each passageway of the plurality of passageways 590 of the inner member 520 such that when adhesive is introduced into the inner member interior lumen 586 at the inner member interior lumen first opening 594, the adhesive is advanced toward the inner member second end 582 and exits from the plurality of passageways 590. The fluid communication between the inner member interior lumen 586 and the plurality of passageways 590 is considered advantageous at least because the plurality of passageways can facilitate distribution of adhesive by spraying adhesive onto two or more diverticula at a treatment portion of the colon.

Atraumatic tip 592 is disposed on the inner member second end 582. As illustrated in FIG. 8, the atraumatic tip 592 is attached to the inner member 520 and defines the inner member second end 582. The atraumatic tip 592 defines a terminating surface 596 such that when the atraumatic tip 592 is attached to the elongate member 510, the terminating surface 596 of the atraumatic tip 592 seals and terminates the inner member interior lumen 586. The atraumatic tip 592 is also tapered and creates an edge to perforate the colon to allow the inner member 520 to pass through the colon and spray the adhesive material over two or more diverticula at a treatment portion of the colon. In the illustrated example, the atraumatic tip 592 is integrally formed with the inner member 520 on the inner member second end 582. Examples of suitable attachment between the atraumatic tip 592 and the inner member include fixed attachment formed with an adhesive bond between the inner member 520 and the atraumatic tip 592 and a mechanical connection formed between the inner member 520 and the atraumatic tip 592. It is noted, though, that the atraumatic tip 592 can include an open end toward the inner member second end 582 and omit the terminating surface 596 such that the inner member interior lumen 586 is in fluid communication with the atraumatic tip 592.

The inner member 520 can be formed of any suitable flexible, or substantially flexible, material, and skilled artisans will be able to select a suitable material for an inner member according to a particular embodiment based on various considerations, including the bodily passage within which the inner member is intended to be used. The material selected for an inner member need only be biocompatible, or able to be made biocompatible, and able to move between a first position and a second position such as shape-memory alloys and/or shape-memory polymers, as described herein. Example materials considered suitable include, but are not limited to, polymers, such as stainless steel, titanium, nickel titanium, nickel titanium alloys (e.g., nitinol), and plastics, such as nylon, polyethylene, and polycarbonate.

While the inner member 520 has been described and illustrated with a length 581 and a diameter 583, an inner member can comprise any suitable length and diameter for a medical device. Skilled artisans will be able to select a suitable length and diameter according to a particular embodiment based on various considerations, including the length and width of the desired bodily passage within which a medical device is intended to be used. An example length for an inner member considered suitable comprises a length between about 1.0 meter to about 1.5 meters, and an example diameter for an inner member considered suitable comprises a diameter between about 0.5 centimeter to about 2.0 centimeters.

FIGS. 6 and 7 illustrate the inner member 520 at a first position and a second position, respectively, while advancing through the elongate member 510 during use.

FIG. 6 illustrates the inner member 520 shown in a first position. In the first position, the inner member 520 is introduced into the elongate member 510 such that the inner member first end 580 is introduced into the first lumen first opening 540 and advances through the first lumen 512 toward the first lumen second opening 542 by use of an external force exerted on an actuator 600 directing the inner member 520 toward the elongate member second end 524. As best illustrated in FIG. 6, the actuator 600 is attached to the inner member 520 towards the inner member second end 580. Once the inner member 520 is advanced to the first lumen second opening 542 by use of the actuator 600, the inner member first end 580 interfaces with the bend 544 of the first lumen 512 such that the bend 544 guides the inner member 520 toward the first lumen second opening 542 and into the internal lumen of the colon (not illustrated in FIG. 6). Once the inner member first end 580 interfaces with the first lumen second opening 542, the atraumatic tip 592 of the inner member 520 perforates the colonic wall to allow the inner member 520 to advance through the colonic wall.

FIG. 7 illustrates the inner member 520 shown in a second position such that a portion of the inner member 520 is exposed into the external environment, including the atraumatic tip 592 and the plurality of passageways 590. After the inner member 520 advances through the colonic wall, the inner member 520 exhibits a curvilinear shape once the inner member 520 is not under pressure due to the inner member 520 having a shape-memory material. As best illustrated in FIG. 7, the shape-memory material allows the inner member 520 to take on a predetermined shape, such as a U-shape, measured at a second angle α₂ relative to the lengthwise axis 511 of the elongate member 510. The predetermined shape of the inner member 520 is considered advantageous at lease because it allows the plurality of passageways 590 to adequately distribute an adhesive, as described below, across two or more diverticula 216 on a treatment portion of the colon. A skilled artisan will be able to determine a suitable second angle and a suitable predetermined shape for the inner member according to a particular embodiment based on various considerations, including the length of the treatment portion of the colon, the number of diverticulum positioned on the treatment portion of the colon, and other considerations. Furthermore, in the second position, the inner member 520 is then advanced until the plurality of passageways 390 are substantially positioned over to two or more diverticula (not illustrated in this figure) on the treatment portion of the colon.

While the medical device 500 has been described and illustrated as using a singular inner member 520, a medical device can have any suitable number of inner members disposed in a first lumen of an elongate member. Skilled artisans will be able to select a suitable number of inner members to place in the first lumen of the elongate member according to a particular embodiment based on various considerations, including the desired bodily passage within which a medical device is intended to be used. Furthermore, while the medical device 500 has been described and illustrated as using a single first lumen 512, a medical device can have any suitable number of lumens used to dispose an inner member or multiple inner members. Skilled artisans will be able to select a suitable number of lumens to place in an elongate member to dispose an inner member or multiple inner members according to a particular embodiment based on various considerations, including the number of sides of the colon an inner member will be administered, the number of the inner member disposed in a lumen, and other considerations. In the illustrated embodiment, the medical device 500 has an elongate member 510 including a singular first lumen 512 where the first lumen 512 disposes a singular inner member 520.

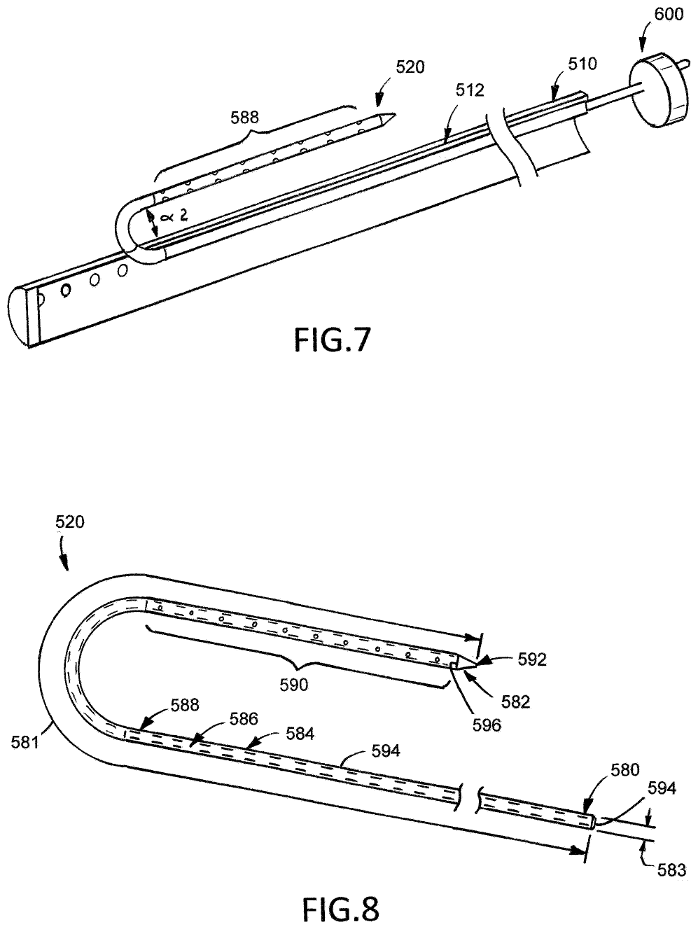

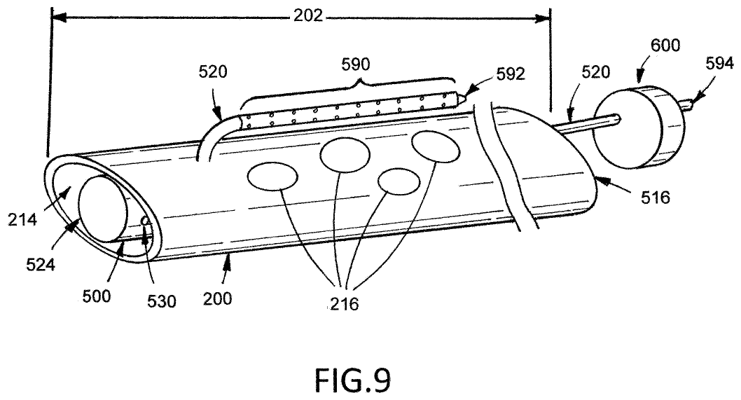

FIG. 9 illustrates the medical device 500 in use while inserted in a treatment portion 202 of a colon 200 affected by diverticulitis. Before the medical device 500 is introduced into the affected colon 200, a balloon catheter including one or more balloons, which is not illustrated in the embodiment, may be introduced into second lumen first opening 550 and advanced through the second lumen 514 toward the second lumen second opening 552. Once the balloon catheter passes through the second lumen second opening 552, the balloon catheter can be introduced into the colon 200 to isolate the treatment portion 202 exhibiting diverticuli. As illustrated in FIG. 9, the medical device 500 is introduced into the treatment portion 202 of the affected colon 200 and depressurizes the treatment portion 202 of the affected colon 200 by collapsing both the treatment portion 202 of the affected colon 200 and two or more diverticula 216 located on the treatment portion 202 of the affected colon 200. As previously described and illustrated in FIGS. 6 and 7, the inner member 520 is advanced through the first lumen 512, passes through the first lumen second opening 542, perforates the colonic wall by use of the atraumatic tip 592, retains its predetermined shape once the inner member 520 enters the internal lumen 214 of the colon 200, and the plurality of passageways 590 of the inner member 520 is positioned over two or more diverticula 216 of the treatment portion 202 of the colon 200. Once the plurality of passageways 590 are substantially positioned over the collapsed diverticuli 216, an adhesive is delivered to the external surface of the treatment portion 202 where the collapsed diverticuli 216 are located. If an adhesive is used during treatment, the adhesive is introduced into the interior lumen opening 592 in the inner member first end 580 via an external container or syringe attached at the inner member first end 580. By exerting an external force on the syringe or the external container containing the adhesive, the adhesive advances through the inner member interior lumen 586 toward the inner member second end 582, exits each passageway of the plurality of passageways 590, and sprays onto the outer surface of the treatment portion 202 of the colon 202. Once the adhesive is sprayed onto the diverticuli 216, the inner member 520 can then be retracted from the colon 202 and into the first lumen 512 by exerting force on the actuator 600 in a direction towards the elongate member first end 524.

The adhesive described in the embodiment can be any suitable adhesive material, and skilled artisans will be able to select a suitable adhesive material according to a particular embodiment based on various considerations, including the type of adhesive needed to attach a material to the outer surface of a treatment portion of the colon where the collapsed diverticuli are located. Examples of adhesive material considered suitable include, but are not limited to, TissuGlu® surgical adhesive (Cohera Medical, Inc., Raleigh, N.C.), Sylys® surgical sealant (Cohera Medical, Inc., Raleigh, N.C.), and CoSeal surgical sealant (Baxter, Deerfield, Ill.). U.S. Pat. No. 9,216,235 to Brownlee et al. for PHOTOACTIVATED CROSSLINKING OF A PROTEIN OR PEPTIDE also describes compositions, and related techniques, considered suitable for use as an adhesive and/or sealant in the inventive methods described herein and is hereby incorporated into this disclosure in its entirety for the purpose of describing compositions suitable for use in steps of applying a material and steps of applying an adhesive, and for describing techniques suitable for use in steps of curing such material when used as an adhesive in relevant methods. Moreover, if a user selects an adhesive that is required to be cured by a light, an external light source, such as a fiber optic wire, may be introduced into the suction lumen of the elongate member to cure the selected adhesive. The inventors have determined that once the external light source is introduced and advanced through the suction lumen toward the elongate member second end, the external light can be applied on the internal lumen of the colon by allowing the light to pass through the plurality of passageways on the elongate member to cure the adhesive material.

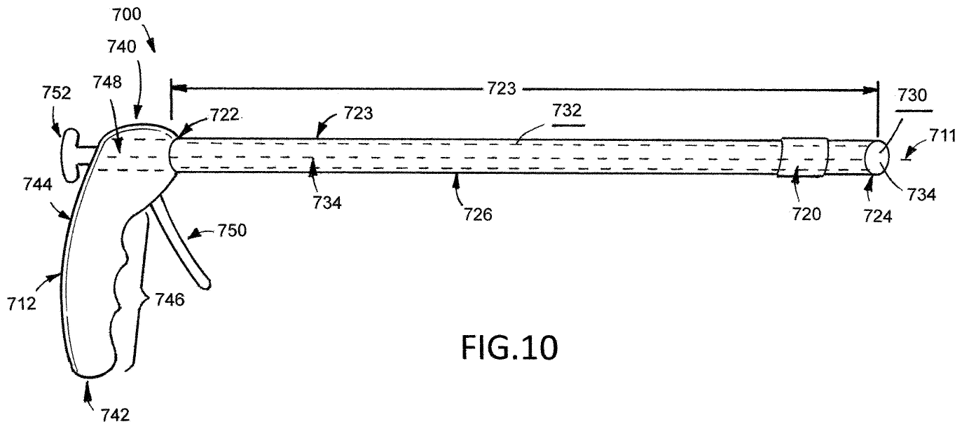

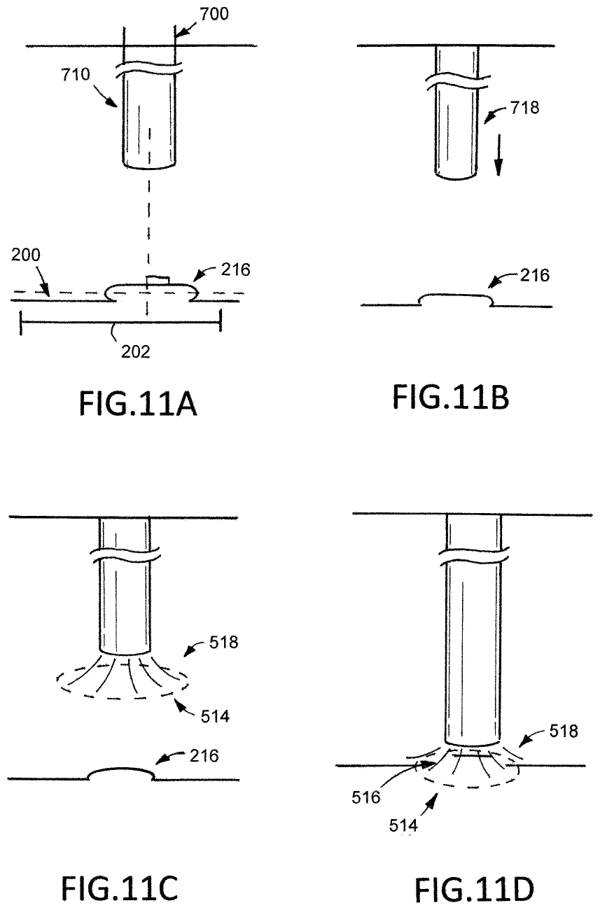

FIGS. 10, 11A, 11B, 11C, and 11D illustrate another example medical device 700 useful in treating diverticulosis to a patient. Medical device 700 comprises an elongate tubular shaft 710, a handle 712, a patch 714, and a deployment system defining a web mechanism 718.

Elongate tubular shaft 710 has an elongate tubular shaft first end 722, an elongate member tubular shaft second end 724, a lengthwise axis 511 extending between the elongate tubular shaft first end 522 and the elongate tubular shaft second end 524, a length 523 extending between the elongate tubular shaft first end 522 and the elongate tubular shaft second end 524, a diameter 525, and an elongate tubular shaft main body 526 extending between the elongate tubular shaft first end 522 and the elongate tubular shaft second end 524. The elongate tubular shaft main body 726 defines an inner surface 728 extending between the elongate tubular shaft first end 722 and the elongate tubular shaft second end 724, an outer surface 730 extending between the elongate tubular shaft first end 722 and the elongate tubular shaft second end 724, and an interior lumen 732 defining an interior lumen first opening 734 and an interior lumen second opening (not illustrated).

First interior lumen 732 extends from the first interior lumen first opening 734 to the first interior lumen second opening and is disposed on the inner surface 728 of the elongate tubular shaft 710 extending along the lengthwise axis 711 of the elongate tubular shaft 710 between the elongate tubular shaft first end 722 toward the elongate tubular shaft second end 724. In the illustrated embodiment, the first interior lumen first opening 734 is disposed on the elongate tubular shaft second end 724, and the first interior lumen second opening is disposed toward the elongate tubular shaft first end 722.

Handle 712 of the medical device 700 has a handle first end 740, a handle second end 742, a handle main body 744, a grip 746, and a second interior lumen 748. As illustrated in FIG. 10, the handle 712 is continuous with the elongate tubular shaft 710, and the handle 712 and the elongate tubular shaft 710 are attached at the handle second end 742 and at the elongate tubular shaft first end 722. The second interior lumen 748 of the handle 712 is disposed toward the handle second end 742 and is continuous with the first interior lumen 732 of the elongate tubular shaft 710. As illustrated in FIG. 10, the grip 746 extends from handle second end 742 toward the handle first end 740. The grip 746 is sized and configured to allow a user, such as surgeon, to adequately hold and manipulate the medical device 700 while in use.

While the elongate tubular shaft 710 has been described and illustrated with a length 723 and a diameter 725, an elongate tubular shaft can comprise any suitable length and diameter for a medical device. Skilled artisans will be able to select a suitable length and diameter according to a particular embodiment based on various considerations, including the length and width of the desired bodily passage within which a medical device is intended to be used. An example length for an elongate tubular shaft considered suitable comprises a length between about 30 centimeters, and an example diameter for an elongate tubular member considered suitable comprises a diameter no larger than 12 millimeters.

Handle 714 also defines a deployment mechanism including a first deployment element 750 and a second deployment element 752. Each of the first and second deployment elements 750, 752 are independent of each other and are used to control either the web mechanism 718 for medical device 700 or the collar 720 of medical device 700′, as described in detail below, to deploy a patch 716 on a treatment portion of a colon. In the illustrated embodiment, the first deployment element 750 is mechanically attached to the collar 720 such that the first deployment element 750 can retract the collar 720 from a first position to a second position when the first deployment element 750 is activated by a user. Similarly, the second deployment element 752 is mechanically attached to the web mechanism 718 such that the second deployment element can extend the web mechanism 718 from a first position to a second position when activated by a user and can retract the web mechanism 718 from the second position to the first position when activated by the user. The mechanical attachment between the first deployment element 750 and the collar 720 are connected in the first interior lumen 734 of the elongate tubular shaft 710 and the second interior lumen 748 of the handle 712. Similarly, the mechanical attachment between the second deployment element 752 and the web mechanism 718 are connected in the first interior lumen 734 of the elongate tubular shaft 710 and the second interior lumen 748 of the handle 712. The mechanical attachments being disposed in the first and second interior lumens 732, 748 are considered advantageous at least because the mechanical attachments are isolated from the external environment to avoid any obstructions when the medical device 700 is inserted into a bodily passage and removed from a bodily passage.

As illustrated in FIGS. 11A, 11B, 11C, and 11D, the web mechanism 718 is disposed toward the elongate tubular shaft second end 724. A portion of the web mechanism 718 extends from the elongate tubular shaft second end 724 toward the elongate tubular shaft first end 722 and connects the web mechanism 718 to the first deployment element 750. Additionally, a portion of the web mechanism 718 also extends out of the elongate tubular shaft second end 724 and folds circumferentially around the outer surface 730 of the elongate tubular shaft 710 at the elongate tubular shaft second end 724. The inventors have determined that the web mechanism 718 is able to fold circumferentially about the outer surface 730 of the elongate tubular shaft 710 at about 180° .

FIGS. 11A, 11B, 11C and 11D illustrate the medical device 700 in use when having a web mechanism 718. Before the medical device 700 is introduced into the bodily passage, the patch 714 is loaded into the web mechanism 718 by folding the patch 714 into the web mechanism 718 to protect and transport the patch material when introduced into the bodily passage. FIG. 11A illustrates the medical device 700 being introduced into a bodily passage to align the elongate tubular shaft 710 of the medical device 700 perpendicularly to one or more diverticula 216 on a treatment portion of an affected colon 200. It is stated, though, the medical device can also be aligned parallel to one or more diverticula 216 on a treatment portion of an affected colon 200. Once the medical device 700 is sufficiently aligned, the user, such as a surgeon, activates a second deployment element 752 to advance the web mechanism 718 toward the elongate tubular shaft second end 724 as shown in FIG. 11B. Once the web mechanism 718 reaches the elongate tubular shaft second end 724 and exits the first interior lumen 734 of the elongate tubular shaft 710, the web mechanism 718 fully expands to allow the patch 714 to fully unfold. Once the patch 714 is unfolded, the user can press and attach the unfolded patch 714 over the collapsed diverticula 216 by manipulating the medical device 700 such that the user exerts force onto the handle 712 directed toward the bodily passage. Once the patch 714 is pressed on to and attached to the collapsed diverticula 216, the user can activate the second deployment element 752 to retract the web mechanism into the first interior lumen 734 of the elongate tubular shaft 710 and remove the medical device 700 from the patient by exert a pulling force on the handle 714 directed away from the bodily passage.

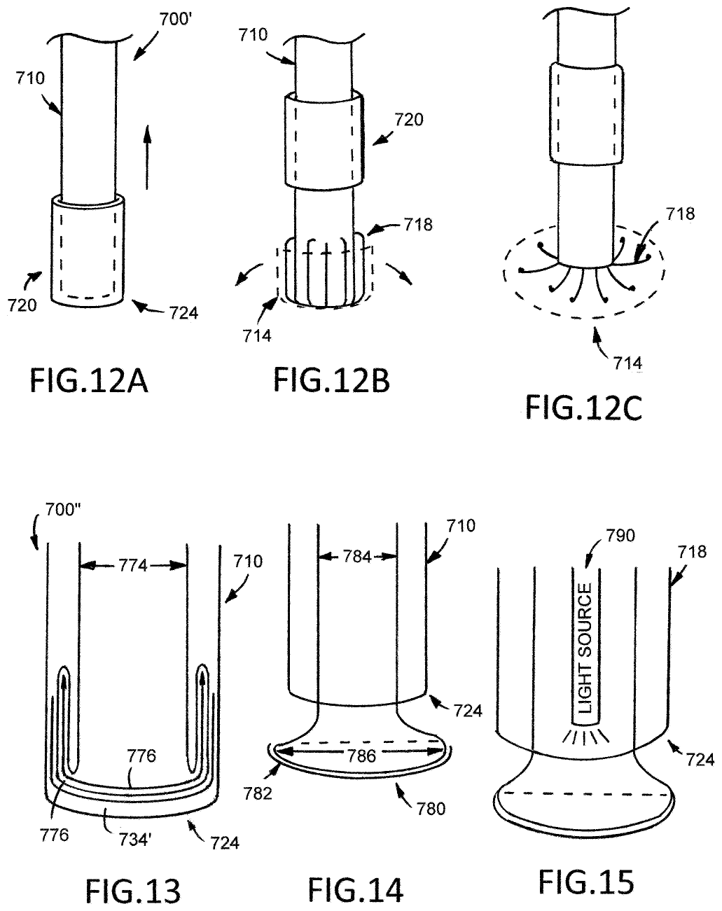

Furthermore, as best illustrated in FIGS. 10 and 12A through 12C, the medical device 700′ has a collar 720 that is disposed between the elongate tubular shaft first end 722 and the elongate tubular shaft second end 724 toward the elongate tubular second end 724. The collar 720 has an inner surface 760 that is sized and configured to interface with the outer surface 730 of the elongate tubular shaft 710 such that collar 720 can move along the length of the elongate tubular shaft from the elongate tubular shaft second end 724 toward the elongate tubular shaft when the first deployment element 750 is activated.

FIGS. 12A, 12B, and 12C illustrate the medical device 700′ in use when having a web mechanism 718 and a collar 720. Before the medical device 700′ is introduced into the bodily passage, the patch 714 is loaded into the web mechanism 718 by folding the patch 714 into the web mechanism 718 to protect and transport the patch 714 when introduced into the bodily passage. However, in the present example, FIG. 12A illustrates the collar 720 at a first portion such that the collar 720 is positioned over the web mechanism 718 and the patch 714 to fold both the web mechanism 718 and the patch 714 over the outer surface 732 of the elongate tubular shaft 710 to protect and transport the web mechanism 718 and the patch 714 when introduced into the bodily passage. As illustrated in FIG. 12B, once the medical device 700′ is introduced into the bodily passage and is sufficiently aligned to one or more diverticula 216 on a treatment portion of an affected colon 200, the user, such as a surgeon, activates a first deployment element 750 to retract the collar 720 from a first position to a second position. The retraction of the collar 720 from the first position the section position allows the web mechanism 718 to expand and allows the patch 714 to unfold completely. Once the patch 714 is unfolded, the user can press and attach the unfolded patch 714 over the collapsed diverticula 216 by manipulating the medical device 700 ‘such that the user exerts force onto the handle 712’ directed toward the bodily passage. Once the patch 714 is pressed on to and attached to the collapsed diverticula 216, the user can remove the medical device 700′ from the patient by exert a pulling force on the handle 712′ directed away from the bodily passage.

FIGS. 13, 14, and 15 illustrate an alternative example of a medical device 700″ that has a balloon mechanism 770. Medical device 700″ is similar to medical device 700 illustrated in FIGS. 10, 11A, 11B, and 11C and medical device 700′ illustrated in FIGS. 12A, 12B, and 12C and described above, except as detailed below. The medical device 700″ includes an elongate tubular shaft 710″, a handle 712″, and a balloon mechanism.

The balloon mechanism 770 has a balloon mechanism first end (not illustrated), a balloon mechanism second end 772, a first diameter 774, and a second diameter 776. The balloon mechanism 770 extends between the elongate tubular shaft first end 722″ to the elongate tubular shaft second end 724″. The first diameter 774 of the balloon mechanism 770 is defined from the balloon mechanism first end toward the balloon mechanism second end 772, and the second diameter 776 of the balloon mechanism is defined on the balloon mechanism second end 776. As illustrated in FIG. 13, the second diameter 776 of the balloon mechanism 770 is greater than the first diameter 774 of the balloon mechanism 770. Furthermore, the balloon mechanism 770 is attached to a deployment element (not illustrated) on the balloon mechanism first end to allow the balloon mechanism 770 to advance between a first position and a second position.

Before the medical device 700″ is introduced into a bodily passage, the patch 714 is loaded into elongate tubular shaft 710″ and is positioned adjacent to the balloon mechanism 770 on the elongate tubular shaft second end 724″ to protect and transport the patch 714 when introduced into the bodily passage. Once the patch 714 is folded and placed in the first interior lumen 734″ of the elongate tubular shaft 710″, a user, such as a surgeon, advances the medical device 700″ into the bodily passage and aligns the medical device 700″ perpendicularly to the collapsed diverticula on the treatment portion of the affected colon, not illustrated. Once aligned, the user activates a deployment element disposed on the medical device 700″ toward the handle 712″ and the elongate tubular shaft first end 722′ such that the balloon mechanism 770 and the patch 714 are simultaneously advanced through the elongate tubular shaft second end 724″ and advanced out of the elongate tubular shaft 710″. Additionally, as illustrated in FIG. 13, a portion of the balloon mechanism 770 extending between the balloon mechanism first end toward the balloon mechanism second end 772 is partially pressurized, but the balloon mechanism second end 772 remains deflated when positioned in the first interior lumen 734″ of the elongate tubular shaft 710″. The different pressurizations in the balloon mechanism 770 are considered advantageous at least because they allow the balloon mechanism 770 to be advanced through the first interior lumen 734″ of the elongate tubular shaft 710″ without any obstructions and expands once the balloon mechanism second end 772 exits the first interior lumen 734″ of the elongate tubular shaft 710″. Once the balloon mechanism second end 772 and the patch 714 exit the first interior lumen 734″ of the elongate tubular shaft 710′, the balloon mechanism second end 772 expands and the patch 714 unfolds. During this stage, the balloon mechanism second end 772 is inflated by use of an external pressurized air supply to allow the second diameter 776 of the balloon mechanism second end 772 to reach its full volume to adequately press and attach the patch 714 to the diverticula. Once the second diameter 772 is completely inflated and the patch 714 is unfolded, the user can press and attach the unfolded patch 714 over the collapsed diverticula by manipulating the medical device 700″ such that the user exerts force onto the handle 712″ directed toward the bodily passage. Once the patch 714 is pressed on to and attached to the collapsed diverticula, the user can remove the air pressure to the balloon mechanism, activate the deployment element to retract the balloon mechanism 772 into the first interior lumen 734″ of the elongate tubular shaft 710″, and remove the medical device 700″ from the patient by exert a pulling force on the handle 712″ directed away from the bodily passage.

As illustrated in FIG. 14, the articulating arm mechanism 780 has an articulating arm mechanism first end (not illustrated), an articulating arm second end 782, a first diameter 784, and a second diameter 786.

The medical devices 700, 700′, 700″ illustrated in the previous embodiments may also comprise an optical fiber 790 or other structure for transmitting light from an internal or external light source. As illustrated in FIG. 15, the medical device 700″ may dispose the optical fiber 790 inside of the balloon mechanism 770 or the articulating arm mechanism 780. Additionally, in medical device 700 or medical device 700′, the optical fiber 790 may be disposed inside the first interior lumen 734 of the elongate tubular shaft 710 and/or the first interior lumen 734 of the elongate tubular shaft 710. The inclusion of the optical fiber 780 is considered advantageous at least because if a user selects an adhesive that is required to be cured by a light, an external light source, such as a fiber optic wire, may be introduced to cure the selected adhesive.

Those with ordinary skill in the art will appreciate that various modifications and alternatives for the described and illustrated examples can be developed in light of the overall teachings of the disclosure. Accordingly, the particular arrangements of elements and steps disclosed are intended to be illustrative only and not limiting as to the scope of the invention, which is to be given the full breadth of the appended claims and any and all equivalents thereof.

What is claimed is:

1. A method of treating diverticulosis in an animal having a colon comprising an external surface having one or more diverticula and defining a lumen, said method comprising the steps of:
clearing said lumen of solid matter;
isolating a treatment portion of said colon, the treatment portion having an axial length;
applying suction at a point within said lumen to depressurize the treatment portion;
applying a material to said external surface across at least one diverticulum of said one or more diverticula, the at least one diverticulum located on a surface of the colon within the axial length of the treatment portion; and
removing the suction.

2. The method of claim 1, wherein the step of isolating a treatment portion of said colon comprises expanding one or more balloons within the lumen to isolate the treatment portion.

3. The method of claim 1, wherein the step of applying suction is performed before the step of applying a material is initiated.

4. The method of claim 1, wherein the step of applying suction is performed until the at least one diverticulum of said one or more diverticula loses residual stress such that the at least one diverticulum collapses.

5. The method of claim 1, wherein the step of applying suction is continued while the step of applying a material is performed.

6. The method of claim 1, wherein the step of applying a material comprises applying a patch of material to said external surface of said colon across the at least one diverticulum.

7. The method of claim 6, wherein the step of applying a material is performed solely through an external approach.

8. The method of claim 7, wherein the external approach comprises surgical access to said external surface.

9. The method of claim 7, wherein the external approach comprises laparoscopic access to said external surface.

10. The method of claim 6, wherein the step of applying a material is performed through a combination of internal and external approaches.

11. The method of claim 10, wherein the step of applying a material includes delivering an adhesive to a location on said external surface from within said lumen of said colon and applying a patch to said external surface at the location through an external approach.

12. The method of claim 1, wherein the step of applying a material comprises applying a first material to said external surface across at least one diverticulum of said one or more diverticula; and
further comprising applying a second material to said external surface across at least one diverticulum of said one or more diverticula.

13. The method of claim 12, wherein the first material comprises an adhesive.

14. The method of claim 13, wherein the step of applying a material is performed prior to the step of applying a second material.

15. The method of claim 14, wherein the second material comprises a hydrogel or a bioremodelable material.

16. The method of claim 14, wherein the second material comprises an extracellular matrix material.

17. The method of claim 16, wherein the extracellular matrix material comprises small intestinal submucosa.

18. The method of claim 14, wherein the second material comprises one of pulmonary ligament, visceral pleura, pericardium, and peritoneum.

19. A method of treating diverticulosis in an animal having a colon comprising an external surface having two or more diverticula and defining a lumen, said method comprising the steps of:
clearing said lumen of solid matter;
isolating a treatment portion of said colon, the treatment portion having an axial length;
applying suction at a point within said lumen to depressurize the treatment portion;
applying an adhesive to said external surface across two or more diverticuli of said two or more diverticuli, the two or more diverticuli located on a surface of the colon within the axial length of the treatment portion;
applying a material to said external surface across the two or more diverticuli;
curing the adhesive; and
removing the suction.

20. A method of treating diverticulosis in an animal having a colon comprising an external surface having two or more diverticula and defining a lumen, said method comprising the steps of:
clearing said lumen of solid matter by administering an enema;
isolating a treatment portion of said colon by inserting a catheter having a balloon into said colon and inflating the balloon within said lumen to isolate the treatment portion from another portion of said colon, the treatment portion having an axial length;
applying suction at a point within said lumen to depressurize the treatment portion;
applying an adhesive to said external surface across two or more diverticuli of said two or more diverticuli, the two or more diverticuli located on a surface of the colon within the axial length of the treatment portion;
applying a material to said external surface across the two or more diverticuli;
curing the adhesive; and
removing the suction.
